(19) United States
(12) Patent Application Publication
(10) Pub. No.: US 2026/0305913 A1
(43) Pub. Date: Oct. 8, 2026

(54) ENDOSCOPY DEVICE HAVING ENHANCED FLUID CONTROL MECHANISMS

(71) Applicant: EvoEndo, Inc., Centennial, CO (US)

(72) Inventors: Alejandro Espinosa, Miami, FL (US); David Castano Galindo, Fort Lauderdale, FL (US)

(21) Appl. No.: 19/480,796

(22) PCT Filed: Apr. 24, 2024
(86) PCT No.: PCT/US2024/025921

§ 371 Date: Oct. 31, 2025

Related U.S. Application Data
(60) Provisional application No. 63/499,681, filed on May 2, 2023.
(60) Provisional application No. 63/499,683, filed on May 2, 2023.
(60) Provisional application No. 63/499,686, filed on May 2, 2023.

(51) Int. Cl. A61B 1/00; A61B 1/015; A61B 1/233; A61M 25/00

(52) U.S. Cl. CPC A61B 1/00068; A61B 1/00119; A61B 1/00128; A61B 1/015; A61B 1/233; A61M 25/0026

(57) ABSTRACT

Systems and methods are disclosed for an endoscope for use in a surgical procedure, e.g., a pediatric trans-nasal endoscopy procedure. The endoscope may include a handle for gripping by a user and a shaft extending from the handle. The shaft may have a working channel extending longitudinally therethrough and a distal region configured to be inserted into a patient. The endoscope may also include a fluid control mechanism on the handle, the fluid control mechanism including a manifold. The manifold may have two or more inlet openings that are configured for receiving a flow of a respective one of air, suction or water, and has a common outlet opening connected to the working channel of the shaft. The endoscope may also include multiple tubes connected to the inlet openings, the tubes connected to each other along their respective lengths but being selectively disconnectable, e.g., peelable, so as to reduce the amount of loose tubing in a surgical setting. Certain tubes may be connectable to a water bottle with a cap having a pressure relief valve to prevent over-pressurization of the water bottle.

### CROSS-REFERENCE TO RELATED APPLICATIONS

This application is a national stage application, filed under 35 U.S.C. § 371, of Internationl Patent Appliction No. PCT/US2024/025921 filed on Apr. 24, 2024, which claims the benefit of U.S. Provisional Application Ser. No. 63/499,681, filed May 2, 2023, U.S. Provisional Application Ser. No. 63/499,683, filed May 2, 2023, and U.S. Provisional Application Ser. No. 63/499,686, filed May 2, 2023, the entire disclosures of each are incorporated herein by reference for all purposes.

### BACKGROUND

Eosinophilic esophagitis (EoE) is an increasingly common chronic inflammatory disease that affects children and adults. Because of its potential to progress to esophageal stricture and the fact that symptoms do not always correlate with degree of eosinophilia, much attention has been paid to repeated assessment of the esophageal mucosa to ensure mucosal healing following treatment. In contrast, the risks, cost and time commitment associated with traditional sedated esophagogastroduodenoscopy (EGD) can be significant and have raised concerns for providers and patients alike. To address these questions, alternative methods are needed to measure esophageal inflammation. In addition to esophagoscopy with biopsies, other technologies such as the Cytosponge, esophageal string test and confocal tethered endomicroscopy have emerged as potential alternatives for assessing mucosal inflammation.

Recent work has led to the development of trans-nasal endoscopy/esophagoscopy (TNE) to assess the esophageal mucosa in adults. In contrast to traditional EGDs, TNE offers advantages, including that it can be performed in an outpatient clinic room, requires no anesthesia or sedation, uses an adult trans-nasal gastroscope that is tolerated by adults and procures samples adequate for assessment of Barrett's Esophagus. However, the endoscopes used in the adult procedures are not appropriate for use in pediatric setting and, in fact, may be too large for many adults.

During a trans-nasal endoscopic procedure, patients may experience physical discomfort due to the endoscope being inserted into the nose, through the sinus cavities and down into the esophagus. This physical discomfort, or even the fear of being uncomfortable, can make trans-nasal endoscopy procedures mentally and emotionally distressing for a patient, too. Because it is desirable to make the procedure mentally and physically easier on the patient, it would be advantageous to optimize the endoscope being used for the procedure.

### SUMMARY

The following presents a simplified summary of the claimed subject matter in order to provide a basic understanding of some aspects of the claimed subject matter. This summary is not an extensive overview of the claimed subject matter. It is intended to neither identify key or critical elements of the claimed subject matter nor delineate the scope of the claimed subject matter. Its sole purpose is to present some concepts of the claimed subject matter in a simplified form as a prelude to the more detailed description that is presented later.

The trans-nasal endoscope described hereinbelow, according to various embodiments, addresses various challenges. For example, the trans-nasal endoscope described hereinbelow, according to various embodiments, provides a device and associated methodology that can be used to adapt TNE to assess the esophageal mucosa, gastric, and duodenal, tracheal, and bronchial mucosa in children and small adults in both a sedated and unsedated manner with a full array of steering and visualization capabilities. The trans-nasal endoscope described hereinbelow, according to various embodiments, provides a scope that minimizes the outer diameter thereof, e.g., to reduce the discomfort to patients, while maximizing the diameter of the working channel, e.g., to provide the largest possible channel through which tools may be introduced, while simultaneously providing enhanced, e.g., four-way, steering capabilities as well as visualization functionality, as will be described more fully below. In various embodiments, the outer diameter of the endoscope shaft may be less than about 4.5 mm, and preferably is about 3.5 mm. In addition, in various embodiments, the diameter of the working channel may have a range of about 1.5 mm to 2.8 mm, and preferably is about 2.0 mm. In certain embodiments, the outer diameter of the endoscope shaft may be between about 4.3 to 4.5 mm, and the diameter of the working channel may be about 2.8 mm.

It is noted that, according to various embodiments, the endoscope described herein may be particularly well-suited for unsedated surgical procedures. Sedation is well-known, in certain circumstances, to present various risks to patients, but is often employed during surgical procedures to prevent a patient from experiencing discomfort or anxiety. By providing an endoscope having, e.g., a minimized outer diameter, more flexible and more steerable distal regions (as will be explained in further detail below) among other advantages described below, patient discomfort and anxiety may be reduced, thereby enabling surgical procedures to be performed in an unsedated, and thus more safe, manner.

It should be recognized that, while the scope set forth herein is described hereinbelow for use in a trans-nasal endoscopy procedure, it may also be employed in a variety of other medical or surgical applications. For example, the scope set forth herein may be employed for use as a nasal endoscope, a trans-nasal esophagoscope, a trans-nasal gastroscope, a trans-nasal duodenoscope, a trans-nasal enteroscope, a triple endoscope, a bronchoscope, a laryngoscope, a trans-nasal gastroscope, an aerodigestive scope, and/or an endoscopic device used to visualize any body cavity into which it would fit, e.g., for examination of a stricture or the like. It should also be recognized that the endoscope described herein may be employed in fetal surgical procedures, and/or in surgical procedures that employ natural orifices, e.g., NOTES or natural orifice transluminal endoscopic procedures, such as trans-orally, trans-anally, trans-vaginally or any other natural orifice. The discussion herein of a pediatric trans-nasal endoscopy procedure is merely exemplary.

In accordance with various embodiments thereof, systems and methods are provided for use in a surgical procedure. In an embodiment, there is provided an endoscope for use in a surgical procedure, e.g., a pediatric trans-nasal endoscopy procedure, that includes a handle for gripping by a user and a shaft extending from the handle. The shaft may have a working channel extending longitudinally therethrough and a distal region configured to be inserted into a patient. The endoscope may also include a fluid control mechanism on the handle, the fluid control mechanism including a manifold. The manifold may have two or more inlet openings that are configured for receiving a flow of a respective one of air, suction or water, and may have a common outlet opening connected to the working channel of the shaft.

In embodiments, the manifold may have three inlet openings, a first inlet opening configured to receive a flow of air, a second inlet opening configured to receive a flow of suction, and a third inlet opening configured to receive a flow of water. The first, second and third inlet openings may have a respective one of a first, second and third valve actuatable by a user to selectively control the flow of its respective one of air, suction or water. Each of the first, second and third valves may have a respective sealing element that is closed in the resting position such that, in the resting position, each valve prevents flow from the respective inlet opening to the outlet opening. Each of the first, second and third valves may also include a spring that biases the valves toward their respective resting positions, each spring having a button mounted thereto that, upon being pressed by a user, overcomes the bias of the spring to move the sealing element from the closed position to an open position at which the respective inlet opening is connected to the outlet opening to thereby permit flow of the respective one of air, suction or water into and through the working channel.

In embodiments, the endoscope may also include a tubing set connected to the handle. The tubing set may include a first air tube connected to the first inlet opening and configured to convey air from an air source towards the working channel. The tubing set may also include a suction tube connected to the second inlet opening and configured to convey suction in the working channel. Still further, the tubing set may also include a water tube connected to the third inlet opening and configured to convey water from a water source towards the working channel.

In further embodiments, the tubing set may also include a second air tube configured to convey air from the air source to the water source so as to pressurize the water source and thereby push water from the water source towards the working channel. The water source may be a water bottle, and the water bottle may have a cap that defines a first connection to the water tube and a second connection to the second air tube.

In still further embodiments, there is provided an endoscope for use in a surgical procedure, e.g., a pediatric trans-nasal endoscopy procedure, that may include a handle for gripping by a user and a shaft extending from the handle. The shaft may have a working channel extending longitudinally therethrough, and may have a distal region configured to be inserted into a patient. In embodiments, the endoscope may also include a tubing set connected to the handle. The tubing set may include a first tube configured to convey a first one of air, suction or water to the working channel, the first tube having a first length. The tubing set may also include a second tube configured to convey a second one of air, suction or water to the working channel, the second tube having a second length. The first and second tube may be connected to each other along their respective lengths by a length of connection material, and the length of connection material may be selectively disconnectable by a user such that respective portions of the first and second lengths remain connected to each other by the connection material and respective other portions of the first and second lengths are disconnected from each other.

In embodiments, the tubing set may also include a third tube having a third length. The third tube may also be connected to one or more of the first and second tubes along their respective lengths by a length of connection material, which may or may not be the same length of connection material that selectively connects the first and second tubes. This length of connection material may also be selectively disconnectable by a user such that respective portions of the first, second and third lengths remain connected by the connection material and respective other portions of the first, second and third lengths are disconnected from each other. In embodiments, the first tube may be configured to convey air towards the working channel, the second tube may be configured to convey suction in the working channel, and the third tube may be configured to convey water towards the working channel.

In embodiments, the endoscope may also be part of a system that includes an air source connected to the first tube and configured to provide the air that is conveyed by the first tube. The endoscope system may also include a suction source connected to the second tube and configured to provide the suction that is conveyed by the second tube. Still further, the endoscope system may also include a water source connected to the third tube and configured to provide the water that is conveyed by the third tube. In embodiments, the water source may be a water bottle, which may have a cap that defines a first connection to the water tube.

In embodiments, the tubing set may also include a fourth tube having a fourth length. The fourth tube may function as a second air tube that is configured to convey air from the air source to a second connection on the cap of the water bottle. As above, the fourth tube may be connected to at least one or more of the first, second and third tubes along their respective lengths by a length of connection material, e.g., which may be the same or different from the above-referenced length of connection material. This length of connection material may be selectively disconnectable by a user such that respective portions of the first, second, third and fourth lengths remain connected by the connection material and respective other portions of the first, second, third and fourth lengths tubes are disconnected from each other.

In still further embodiments, the tubing set may also include a video connector cable configured to conveys signals or data to an external video display device, the video connector cable having a cable length. The video connector cable may be connected to one or more of the first and second tubes along their respective lengths by a length of connection material. The length of connection material may be selectively disconnectable by a user such that respective portions of the first, second and cable lengths remain connected by the connection material and respective other portions of the first, second and cable lengths are disconnected from each other. Additionally or alternatively, the tubing set having the video connector cable may also include the above-referenced third and fourth tubes, and the length of the video connector cable may also be selectively disconnectable therefrom.

In still other embodiments, there is provided an endoscope for use in a surgical procedure, e.g., a pediatric trans-nasal endoscopy procedure, that includes a handle for gripping by a user and a shaft extending from the handle. The shaft may have a working channel extending longitudinally therethrough and a distal region configured to be inserted into a patient. The endoscope may also include a water bottle having a bottle cap, the bottle cap having a first connection. A water tube may be connected to the first connection of the bottle cap and to the handle so as to provide water from the water bottle to the working channel. The bottle cap may have a pressure relief valve so as to prevent over-pressurization within the water bottle, the water tube or the working channel.

In embodiments, the endoscope may also include a pressurizing air tube connected to a second connection on the bottle cap. The pressurizing air tube may be configured to convey pressurized air to the interior of the water bottle so as to push water from the water bottle through the water tube and into the working channel.

In embodiments, the endoscope may also include an air source configured to provide pressurized air to the interior of the water bottle via the pressurizing air tube. In embodiments, the endoscope may also include an air supply tube connected to the air source and connected to the pressurizing air tube within the handle, the air supply tube also configured to provide air to the working channel. The handle may include a fluid control mechanism that controls the flow of water from the water tube into the working channel, and that may control the flow of air from the air supply tube to the working channel.

In embodiments, the endoscope may also include a suction tube connected to the handle and to a suction source. The fluid control mechanism may also control the flow of suction through the suction tube and through the working channel. The fluid control mechanism may also include a first valve actuatable by a user to selectively control the flow of air through the air supply tube into the working channel. The fluid control mechanism may also include a second valve actuatable by a user to selectively control the flow of suction through the suction tube and the working channel. The fluid control mechanism may also include a third valve actuatable by a user to selectively control the flow of water through the water tube and into the working channel. Each of the first, second and third valves may include a spring that biases the valves closed in a resting position. Each of the first, second and third valves may also include a button that, upon being pressed by a user, overcomes the bias of the spring to open the valve and thereby permit flow of the respective one of air, suction or water into and through the working channel. In embodiments, the water tube, the air supply tube, the pressurizing air tube and the suction tube may be connected to each other as a tubing set.

In embodiments, the bottle cap may include a water suction tube that extends into the water bottle. The water suction tube may be adjustable such that the bottle cap fits water bottles of varying heights. The first and second connections of the bottle cap may comprise barbed connections.

It should be noted, of course, that to the extent that images, e.g., image signals, image data, etc., are described herein, it will be understood that such also refers to video, e.g., video signals, video data, etc., and that the description of the image signals is intended to include single images, still images, video images, etc. without limitation.

### DRAWINGS

FIG. 1 shows a schematic representation of a trans-nasal endoscope that includes a flexible endoscope shaft, in accordance with various embodiments.

FIG. 2 is a perspective view of the handle of the trans-nasal endoscope, in accordance with various embodiments.

FIG. 3 is a perspective, cut-away view of the handle in the vicinity of a bifurcation region, with most of the features of the handle being hidden or shown in phantom so as not to obscure the features shown, in accordance with various embodiments.

FIG. 4 is another perspective, cut-away view of the handle in the vicinity of the bifurcation region, but enlarged in comparison to FIG. 3 so as to show additional details of an instrument port, in accordance with various embodiments.

FIG. 5 is a perspective view of some components of an AWS control mechanism with the handle being hidden from view, in accordance with various embodiments.

FIGS. 6A and 6B are top and side cross-sectional views, respectively, of a valve manifold, in accordance with various embodiments.

FIG. 7 is a side view showing a strain relief mechanism provided at a location at which the AWS tubing set enters the handle, in accordance with various embodiments.

FIG. 8 is a perspective view of one end of an AWS tubing set, in accordance with various embodiments.

FIG. 9 is a top perspective view of a water bottle cap for a water source, in accordance with various embodiments.

FIG. 10 is a front perspective, cross-sectional view of a tubing set, in accordance with various embodiments.

### DETAILED DESCRIPTION

Reference will now be made in detail to specific embodiments illustrated in the accompanying drawings. In the following detailed description, numerous specific details are set forth to provide a thorough understanding. However, it will be apparent to one of ordinary skill in the art that embodiments may be practiced without these specific details. In other instances, known methods, procedures, components, circuits, and networks have not been described in detail so as not to unnecessarily obscure aspects of the embodiments.

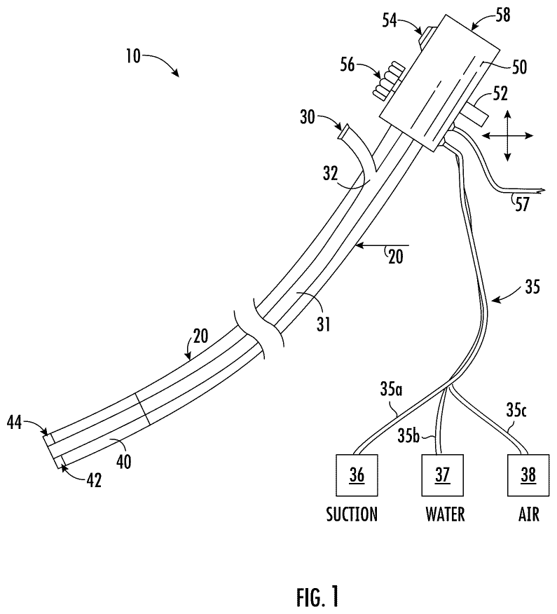

FIG. 1 is a schematic diagram of a trans-nasal endoscope 10, according to one example embodiment, illustrating some of the various features thereof. As mentioned previously, while the example embodiments set forth hereinbelow are described as an endoscope that is suitable for trans-nasal insertion into a patient, and is particularly well-suited for trans-nasal insertion, and particularly unsedated insertion, into a child or small adult, it is understood that this is merely one example embodiment, and that the description hereinbelow of a trans-nasal endoscope does not preclude the use of the device in other types of procedures and for other types of patients. It should be noted that FIG. 1 is merely schematic, and thus the shape and position of the various features illustrated therein are merely exemplary. Additional figures, illustrating specific embodiments of the various features and functionality, will be provided in further detail below.

In the embodiment shown schematically in FIG. 1, the trans-nasal endoscope 10 includes a flexible endoscope shaft 20. The flexible endoscope shaft 20 has a working channel 31. The working channel 31 extends longitudinally from a distal end 40 of the endoscope shaft 20 proximally towards a handle 50 located at or near the proximal end of the trans-nasal endoscope 10. At, within or near the handle 50, the working channel 31 has a bifurcation region 32. Proximal to the bifurcation region 32, the working channel 31 splits into two channels. A first portion of the working channel 31 proximal to the bifurcation region 32 extends towards an instrument insertion port 30 suitable for, e.g., conducting a biopsy therethrough. The instrument insertion port 30 allows an instrument, e.g., a pediatric nasal endoscope biopsy forceps or other medical device, to be inserted through the bifurcation region 32 and to, and past, the distal end 40 of the endoscope shaft 20 so as to perform a procedure, e.g., a biopsy procedure, on tissue located at or near to the distal end 40 of the endoscope shaft 20.

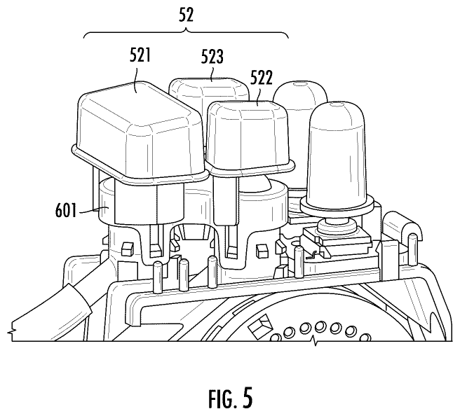

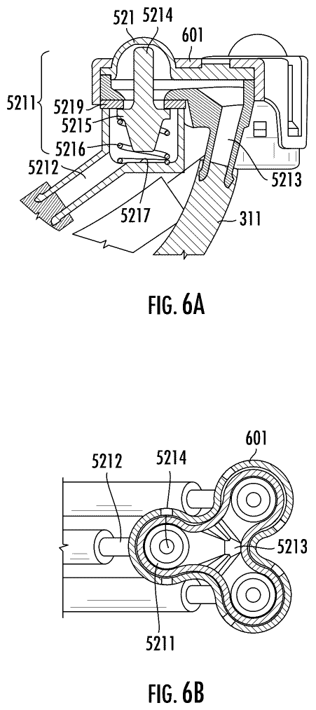

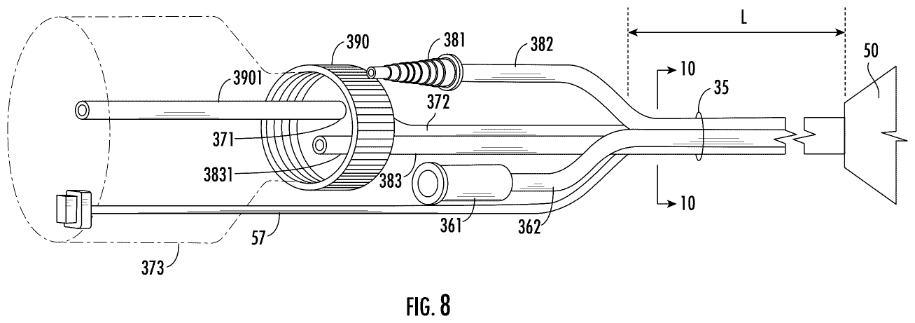

A second portion of the working channel 31 proximal to the bifurcation regions 32 extends towards an air, water and suction (AWS) control mechanism 52. The AWS control mechanism 52 includes various valves (not shown in this view, but shown and described in connection with, e.g., FIGS. 5, 6A and 6B below, and additional details of which are shown and described in Applicant's co-pending U.S. patent application Ser. No. 18/108,558 filed on Feb. 10, 2023, the disclosure of which is hereby incorporated by reference herein in its entirety) that allow selective connection of the working channel 31 to the AWS tubing set 35. The AWS tubing set 35 may include one or more flexible tubes (shown and described separately and in greater detail below in connection with, e.g., FIGS. 8 and 10). The AWS tubing set 35 may be connected to a water source 37 for supplying water through the working channel 31, to a suction source 36 for supplying suction through the working channel 31, and/or to an air source 38 for supplying air through the working channel 31, depending upon a user's selection via the AWS control mechanism 52. More specifically, the AWS control mechanism allows a user to direct one or more of air, suction or water through, e.g., the bifurcation region 32 and to, and past, the distal end 40 of the endoscope shaft 20 so as to enable their use during the performance of a procedure on tissue located at or near to the distal end 40 of the endoscope shaft 20.

The distal end 40 of the endoscope shaft 20 also includes an illumination source 42 to provide light at the distal tip 40. In embodiments, the illumination source 42 may be connected to and at least partially controllable by an electronics control module 54 located in the handle 50. The distal end 40 of the endoscope shaft 20 also includes an image capture device 44 to convey image or video signals related to the region of the distal end 40 of the endoscope shaft 20. In embodiments, the image capture device 44 may also be connected to and at least partially controllable by the electronics control module 54 located in the handle 50. The handle 50 may also include a shaft steering mechanism 56 to control or steer the lateral displacement at the distal end 40 of the endoscope shaft 20. In addition, the handle 50 may include a video display output 57, which may be connected to and output image data to a separate image or video display or control unit (not shown in this view).

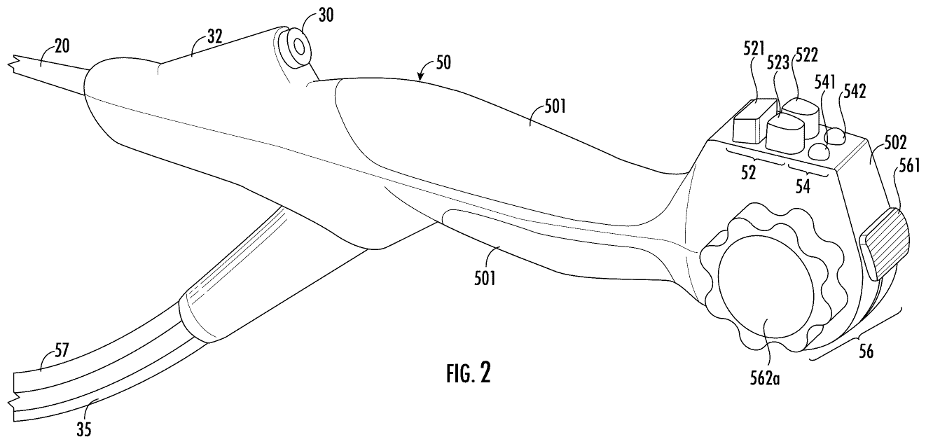

FIG. 2 is a perspective view of an example embodiment of the handle 50 of the trans-nasal endoscope 10. In this embodiment, the features and functionality that were shown schematically in FIG. 1 are provided in more detail, showing additional advantages thereof. For example, in this embodiment, the handle 50 of the trans-nasal endoscope 10 includes a gripping region 501 sized and contoured to fit comfortably in a user's hand. Located distally relative to the gripping region 501 is the bifurcation region 32. From the distalmost end of the bifurcation region 32 extends the flexible endoscope shaft 20, having a portion of the working channel 31 extending therethrough. The working channel 31 extends from the distal end 40 of the endoscope shaft 20, and splits into two channels in the bifurcation region 32. A first portion of the working channel 31 extends towards the instrument insertion port 30, which is suitable for receiving an instrument, e.g., a pediatric nasal endoscope biopsy forceps or other medical device, therethrough. The second portion of the working channel 31 proximal to the bifurcation region 32 extends proximally through the interior of the gripping region 501.

Proximal to the gripping region 501 is a control region 502 sized and shaped to extend beyond the heel of the user's hand when the palm of the user's hand is gripping the gripping region 501, enabling the control features positioned on the control region 502 to be engaged by the user's second hand when the user's first hand is gripping the gripping region 501.

In the embodiment shown in FIG. 2, the control region 502 has various control features positioned thereon. For example, the control region 502 has the AWS control mechanism 52. As set forth above, the AWS control mechanism 52 includes various features, e.g., buttons, valves, etc., that allow selective connection of the air, water and suction supply sources 36, 37, 38 to the working channel 31 via respective flexible tubes of the AWS tubing set 35. In the embodiment shown in FIG. 2, the AWS control mechanism 52 includes an air supply control button 521. The air supply control button 521 functions to selectively connect the air source 38 to the working channel 31, as will be described in greater detail below in connection with, e.g., FIGS. 5, 6A and 6B.

In the embodiment shown in FIG. 2, the AWS control mechanism 52 also includes a water supply control button 522. The water supply control button 522 functions to selectively connect the water source 37 to the working channel 31, as will be described in greater detail below in connection with, e.g., FIGS. 5, 6A and 6B.

Still further, in the embodiment shown in FIG. 2, the AWS control mechanism 52 includes a suction supply control button 523. The suction supply control button 523 functions to selectively connect the suction source 36 to the working channel 31, as will be described in greater detail below in connection with, e.g., FIGS. 5, 6A and 6B.

In the embodiment shown in FIG. 2, the control region 502 also has the electronics control mechanism 54. As set forth above, the electronics control mechanism 54 includes various features, e.g., buttons, electrical connections, etc., that allow selective operation of, e. g, the image capture device 44 and/or the illumination device 44 located at the distal end 40 of the endoscope shaft 20. In the embodiment shown in FIG. 2, the electronics control mechanism 54 includes a first, e.g., a white balance control, button 541. In this embodiment, the white balance control button 541 functions to selectively control a white balancing operation by sending a corresponding signal to an image or video control unit (not shown), as is shown and described more fully in Applicant's co-pending U.S. patent application Ser. No. 18/108,564 filed on Feb. 10, 2023, and in Applicant's co-pending U.S. patent application Ser. No. 63/499,686 filed on May 2, 2023, the disclosure of both being hereby incorporated by reference herein in their entirety.

In the embodiment shown in FIG. 2, the electronics control mechanism 54 also includes a second, e.g., an image capture control, button 542. In this embodiment, the image capture control button 542 functions to selectively control the capture of image or video signals sent by the image capture device 44, e.g., such as by providing a signal to an image or video display or control unit (not shown), as is shown and described more fully in Applicant's co-pending U.S. patent application Ser. No. 18/108,564 filed on Feb. 10, 2023, and in Applicant's co-pending U.S. Patent Application Ser. No. 63/499,686 filed on May 2, 2023.

In the embodiment shown in FIG. 2, the control region 502 also has the shaft steering mechanism 56. As set forth above, the shaft steering mechanism 56 includes various features, e.g., knobs, rollers, etc., that allow a user to control or steer the lateral displacement at the distal end 40 of the endoscope shaft 20. In the embodiment shown in FIG. 2, the shaft steering mechanism 56 includes a first knob 561 for controlling a first movement of the distal end 40 of the endoscope shaft 20. FIG. 2 also illustrates the shaft steering mechanism 56 including opposing roller knobs 562a, 562b (knob 562b being hidden from view in FIG. 2, but being located on the opposite side of the handle 50) for controlling additional movements of the distal end 40 of the endoscope shaft 20. Additional features and functionality of the shaft steering mechanisms 56 are shown and described more fully in Applicant's co-pending U.S. patent application Ser. No. 18/108,562 filed on Feb. 10, 2023, and in Applicant's co-pending U.S. Patent Application Ser. No. 63/499,683 filed on May 2, 2023, the disclosure of both being hereby incorporated by reference herein in their entirety.

In addition, in the embodiment shown in FIG. 2, the handle 50 of the trans-nasal endoscope 10 includes a connection to the AWS tubing set 35. As set forth above, the AWS tubing set includes various flexible tubes that connect to the suction source 36, the water source 37 and the air source 38, as will be described in greater detail below in connection with, e.g., FIGS. 8 and 10. Still further, the handle 50 includes a connection to a video display output 57, e.g., for connecting to and outputting image data to a separate video display or control unit (not shown in FIG. 2). In the embodiment shown in FIG. 2, the video display output 57 is bundled together with the AWS tubing set 35, as will be described in greater detail below in connection with, e.g., FIGS. 8 and 10.

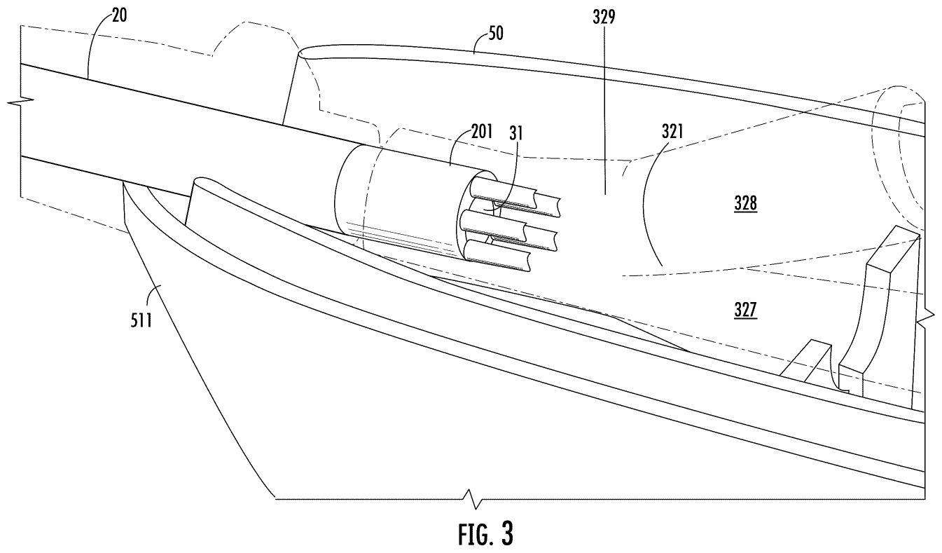

FIG. 3 is a perspective, cut-away view of the handle 50 in the vicinity of the bifurcation region 32, with most of the features of the handle 50 being hidden or shown in phantom so as not to obscure the features shown. In the embodiment shown in FIG. 3, the proximal-most end 201 of the endoscope shaft 20 extends slightly proximal relative to the distal-most end 511 of the handle 50. The working channel 31 which extends longitudinally through the shaft 20 opens into a bifurcation fitting 329. The bifurcation fitting 329 is sealingly fitted onto the end 201 of the shaft 20. The bifurcation fitting 329 functions to split the working channel 31 into two channels within the handle 50.

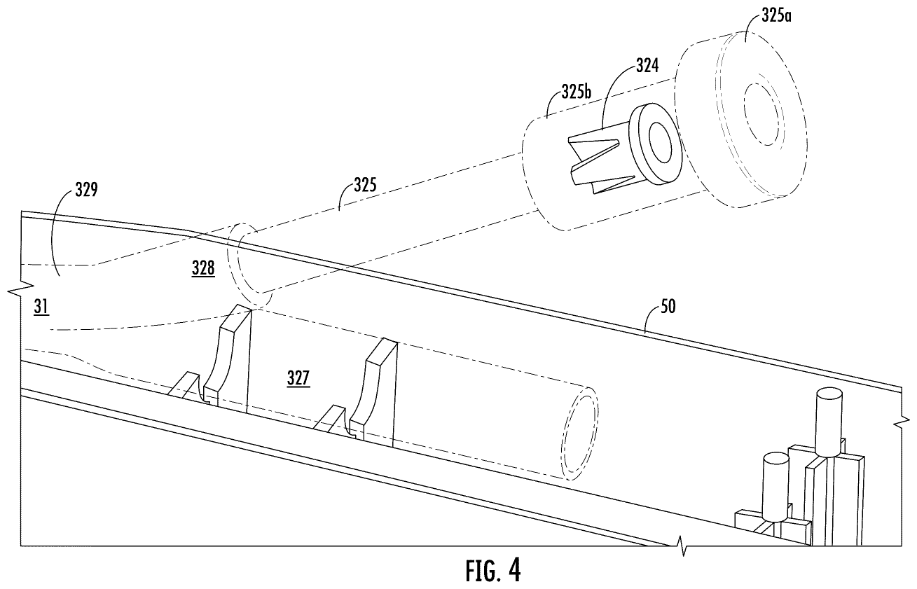

The first channel 328 of the bifurcation fitting 329, in the embodiment shown, angles upwardly towards an instrument port, as will be shown and described in greater detail in connection with FIG. 4. The second channel 327 of the bifurcation fitting 329, in the embodiment shown, extends proximally from the working channel 31 towards the proximal end of the handle 50, where it communicates with a fluid control mechanism, as will be shown and described in greater detail in connection with, e.g., FIGS. 6A and 6B. Of course, it should be recognized that the bifurcation fitting 329 could connect to still other channels if desired, e.g., additional instrument ports or the like, or it could connect to the channels described hereinabove in various different orientations, depending on, e.g., the configuration of the handle 50 among other factors.

FIG. 4 is another perspective, cut-away view of the handle 50 in the vicinity of the bifurcation region 32, but enlarged in comparison to FIG. 3 so as to show additional details of the various components of the handle 50, but again with most of the features of the handle 50 being hidden or shown in phantom so as not to obscure the features shown. Specifically, it can be seen in FIG. 4 that the first channel 328 of the bifurcation fitting 329 connects to an instrument port 325. The instrument port 325 extends from the bifurcation fitting 329 within the handle 50 through an opening in the handle so as to provide a proximal end region 325a that is outside of the handle 50. The proximal end region 325a of the instrument port 325 includes a valve housing 325b in which resides an instrument port valve 324.

The instrument port 325 allows a surgical instrument (not shown) to be introduced into the proximal end region 325a, through the second channel 328 and into and through the working channel 31. The surgical instrument that is introduced and inserted through the working channel 31 via the instrument port 325 may be any conceivable surgical instrument that is sized and shaped to fit therethrough. For example, the instrument port 325 may be configured to receive a biopsy forceps instrument therethrough and into the working channel so as to perform a biopsy procedure on the patient's tissue at or near the distal end 40 of the shaft 20.

In various embodiments, the instrument port valve 324 positioned in the valve housing 325a at the proximal end of the instrument port 325 is configured to prevent the ingress of any foreign matter into the working channel 31 prior to, or during, a surgical instrument being inserted therethrough. In addition, the instrument port valve 324 also functions to prevent the flow of any fluids, e.g., air, water or suction, that may be inside the working channel 31 from escaping through the first channel 328 and out the proximal end region 325a of the instrument port 325. Advantageously, the instrument port valve 324 may be configured to prevent the escape of any such fluids, e.g., air, water or suction, that may be inside the working channel 31 when a surgical instrument is present within the first channel 328. Still further, the instrument port valve 324 may be configured to prevent the escape of any such fluids, e.g., air, water or suction, that may be inside the working channel 31 when no surgical instrument is present within the first channel 328. In some embodiments, a combination of valves may be employed, e.g., an instrument seal for sealing in the presence of an instrument and/or a separate zero seal for sealing the opening when no instrument is present therewithin. In some embodiments, the instrument port valve 324 may be configured as, e.g., a quad-port valve or “double-duckbill” valve, so as to provide a single valve that performs the functions of both an instrument seal, e.g., for sealing in the presence of an instrument, and a zero seal, e.g., for sealing the opening when no instrument is present therewithin.

As set forth above, in various embodiments, the handle 50 includes a bifurcation fitting 329 also having a second channel 327 configured to selectively convey at least one of air, suction or water into the working channel 31. As mentioned in connection with FIG. 2, the control region 502 of the handle 50 has an AWS control mechanism 52 that includes various features, e.g., buttons, valves, etc., that allow such selective connection of the air, water and suction supply sources 36, 37, 38 to the working channel 31 via the second channel 327 of the bifurcation fitting 329.

FIG. 5 is a perspective view (with various components, such as the walls of the handle 50, being hidden so as not to obscure the features shown) of, among other components, an AWS control mechanism 52, according to an embodiment. FIG. 5 illustrates the AWS control mechanism 52 having a control manifold 601 on which are mounted various buttons that allow a user to selectively control the flow of air, suction and water into the working channel 31. In this embodiment, the AWS control mechanism 52 includes an air supply control button 521 that functions to selectively connect an air source, e.g., the air source 38 shown in FIG. 1, to the working channel 31. The AWS control mechanism 52 also includes a water supply control button 522 that functions to selectively connect a water source, e.g., the water source 37 shown in FIG. 1, to the working channel 31. Still further, the AWS control mechanism 52 may also include a suction supply control button 523 that functions to selectively connect a suction source, e.g., the suction source 36 shown in FIG. 1, to the working channel 31.

FIGS. 6A and 6B are top and side cross-sectional views, respectively, of the AWS control mechanism 52, illustrating some of the internal components of an AWS control mechanism 52, according to various embodiments. For example, FIGS. 6A and 6B illustrate the internal components of the air supply control button 521 that functions to selectively connect an air source, e.g., the air source 38 shown in FIG. 1, to the working channel 31. As shown, the air supply control button 521 is part of an air supply control valve 5211. The air supply control valve 5211 includes an air inlet 5212 that is connected to a source of air (not shown here) such as the air supply source 38 shown in FIG. 1. In addition, the air supply control valve 5211 may be connected to a valve manifold 601 which has a common outlet 5213 connected to a tube 311 that is connected to the working channel 31 (not shown here). Still further, the air supply control valve 5211 may include an air valve sealing element 5214 that moves between closed and open positions.

FIG. 6A illustrates the air supply control valve 5211 in the closed position. In this closed position, a sealing ridge 5215 of the air valve sealing element 5214 is seated against a sealing face 5219, such that it seals these two surfaces and thereby prevents air that is entering the air supply control valve 5211 via the air inlet 5212 from exiting the air supply control valve 5211 and from entering the valve manifold and the common outlet 5213.

As further shown in FIG. 6A, the air supply control button 521 engages the air valve sealing element 5214. In addition, the air supply control valve 5211 includes an air valve spring 5216 that is interposed between the air supply control button 521 and a ridge 5217 on the body of the air supply control valve 5211. In this configuration, the air valve spring 5216 biases the air supply control button 521 and the air valve sealing element 5214 upwardly. In the absence of any force applied to the air valve spring 5216, the air supply control valve 5211 remains in this resting position, in which the air valve sealing element 5214 keeps the air supply control valve 5211 closed. By the air valve sealing element 5214 keeping the air supply control valve 5211 closed in this resting position, air is prevented from flowing from the air source through the air supply control valve 5211 and into the working channel 31 of the shaft 20 via the outlet 5213 of the manifold 601.

Although hidden from view in FIG. 6A, the water supply button 522 and the suction supply button 523 of the AWS control mechanism 52 may have internal components similar to those described hereinabove for the air supply button 521, and may thus function to selectively connect a water or suction source, e.g., the water source 37 or the suction source 36 shown in FIG. 1, to the working channel 31 in the same way as described above for the air supply button 521.

In order to operate the AWS control mechanism 52 and thereby permit flow from the respective source of air, suction or water into and through the working channel 31, a user may press the buttons so as to open the valves. For example, in the embodiment shown in FIG. 6A, in order to permit flow from the air source into and through the working channel 31, a user may press the air supply control button 521 downwardly with enough force to overcome the biasing force provided by the air valve spring 5216. Once the biasing force of the air valve spring 5216 is overcome, the air valve sealing element 5214 is moved downwardly until the sealing ridge 5215 of the air valve sealing element 5214 is spaced apart from the sealing face 5219. Once the sealing ridge 5215 of the air valve sealing element 5214 is spaced apart from the sealing face 5219, air entering the air valve inlet 5212 is now free to flow out of the air supply control valve 5211 and out of the common outlet 5213. Still further, and although hidden from view in FIG. 6A, in embodiments in which the water and suction supply buttons 522, 523 of the AWS control mechanism 52 have internal components similar to those described hereinabove for the air supply button 521, the water and suction supply buttons 522, 523 may thus function to selectively open the water and suction supply control valves, respectively, in the same way as described above for the air supply button 521.

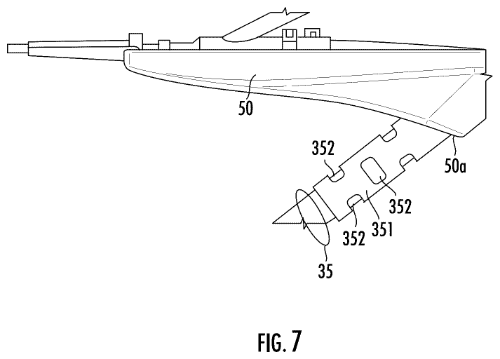

As set forth above, in embodiments, the various valves, e.g., the air supply control valve 521, the water supply control valve 522 and the suction supply control valve 523 of the AWS control mechanism 52, each have inlet openings that are connected to respective external air, water and suction supply sources 37, 38, 39. These respective inlet openings are connected to respective flexible tubes, which, as shown in FIG. 1, may collectively form an AWS tubing set 35 that connect to the handle 50 at a common opening in the handle 50. FIG. 7 is a side view that illustrates such an AWS tubing set 35 connecting to the handle 50 at a common opening of the handle 50a, according to an embodiment. FIG. 7 shows a strain relief mechanism 351 extending through the opening of the handle 50. In an embodiment, the strain relief mechanism 351 is disposed around a portion as the AWS tubing set 35 that is within the interior of the handle, as well as around a portion of the AWS tubing set 35 that extends outside of the handle 50. In this way, the strain relief mechanism 351 may provide some additional reinforcing material that keeps the various tubes of the AWS tubing set 35 from overly stressing, and potentially breaking, the material of the handle 50 at the location where the AWS tubing set 35 exits the handle 50. Advantageously, the strain relief mechanism 351 may also define voids or gaps 352 therein. These voids or gaps 352 may provide the strain relief mechanism 351 with some additional flexibility so that it may be more apt to bend when the AWS tubing set 35 is moved relative to the handle 50, which provides an additional measure of relief helping to prevent the various tubes of the AWS tubing set 35 from overly stressing, and potentially breaking, the material of the handle 50 at the location where the AWS tubing set 35 exits the handle 50.

Additionally or alternatively, it should be recognized that, according to various embodiments, the AWS tubing set 35 may connect to the handle 50 at the common opening of the handle 50a in various different ways. For example, the AWS tubing set 35 could be reusable by having the AWS tubing set 35 be selectively connectable and/or disconnectable from the handle 50 at, e.g., the location 50a. The AWS tubing set 35 may be selectively connectable and/or disconnectable from the handle 50 by employing, e.g., a screw-type fitting (not shown) at the location 50a, and/or by employing a magnetic-type fitting (not shown) that guides and secures the end of the AWS tubing set 35 into the location 50a of the handle 50. It should be recognized that such embodiments may enable both the fluid tubes, e.g., the suction tube 362, the water tube 372, the first air tube 382 and the second air tube 383, as well as the electrical connectors, e.g., video display output cable 57, to be selectively connectable and/or disconnectable to their respective mating tubes/connectors within the handle 50. Such a fitting at the location 50a may also include one or more valves, e.g., one-way valves (not shown) to prevent the backflow of certain fluids through the respective tubes. It should also be recognized that a similar type of fitting arrangement, e.g., permitting reusability, may be employed at the connection between the AWS tubing set 35 and the bottle cap 390.

Of course, it should be recognized that, according to still other embodiments, the various tubes that connect the air, water and suction sources to their respective air, water and suction inlet openings need not connect to the handle 50 at a common opening, but may instead connect to the handle 50 at different locations, and that the stress relief mechanism 351 described hereinabove may be employed at one or more of these different locations. Still further, it should be recognized that, according to still other embodiments, while the stress relief mechanism 351 is described hereinabove in connection with an AWS tubing set 35 having various tubes that connect the air, water and suction sources to their respective air, water and suction inlet openings, the stress relief mechanism 351 may additionally or alternatively be employed for tubing sets having any number and types of tubes, e.g., an AWS tubing set that also includes additional air tubes and/or video or electrical connections. More specifically, the stress relief mechanism 351 may additionally or alternatively be employed for relieving the stress on connections to the handle 50 from any types or numbers of tubing and/or electrical connections that extend from the handle 50.

FIG. 8 is a perspective view of the opposite end of an AWS tubing set 35, according to an embodiment thereof. In this embodiment, the AWS tubing set 35 includes a suction tube 362. One end of the suction tube 362 includes a connection fitting, e.g., suction tube connection fitting 361, that is configured to mate with a corresponding connection fitting on a suction source, e.g., suction source 36 shown in FIG. 1, which may be any conceivable suction supply such as a standard hospital suction connection, a suction tank or the like. The other end of the suction tube 362 is configured to be connected to the inlet opening of the suction supply control mechanism associated with the suction control button 523, as mentioned above.

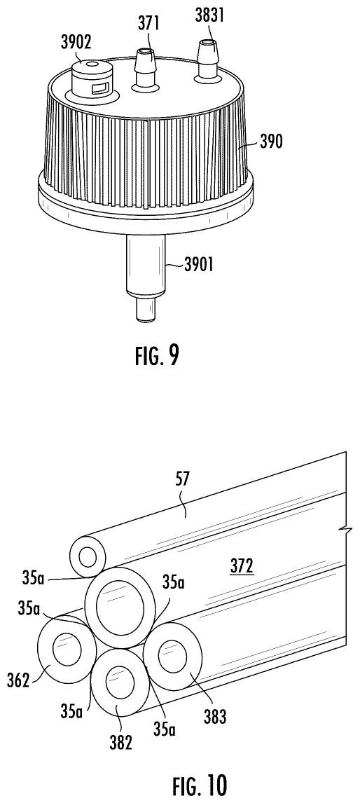

Likewise, FIG. 8 illustrates a water tube 372. One end of the water tube 372 includes a connection fitting, e.g., water tube connection fitting 371, that is configured to mate with a corresponding connection fitting on a water source, e.g., water source 37 shown in FIG. 1. Water source 37 may be any conceivable water supply such as a standard hospital water connection, a water tank or the like. In an embodiment, the water source 37 may be a water bottle 373, which is shown in phantom in FIG. 8. The water bottle 373 may have a water bottle cap 390 configured to be selectively sealed onto the water bottle 373, the water bottle cap 390 having the water tube connection fitting 371 thereon such that the water tube 372 may be selectively connected thereto. The other end of the water tube 372 is configured to be connected to a water inlet of the water supply control valve, as described above in connection with FIGS. 5 and 6A. Additional details and embodiments of the water bottle cap 390 are shown and described below in connection with FIG. 9.

FIG. 8 also illustrates a first air tube 382. One end of the first air tube 382 includes a connection fitting, e.g., first air connection fitting 381, that is configured to mate with a corresponding connection fitting on an air source, e.g., air source 38 shown in FIG. 1. The air source 38 may be any conceivable air supply such as a standard hospital air connection, an air tank or the like. The other end of the first air tube 382 is configured to be connected to the air inlet 5212 of the air supply control valve 5211, as shown and described hereinabove in connection with FIG. 6A. Still further, FIG. 8 illustrates a second air tube 383. One end of the second air tube 383 includes a connection fitting, e.g., second air connection fitting 3831, that may be disposed on the bottle cap 390, as shown and described in greater detail below in connection with FIG. 9. A second end of the second air tube 383 may also extend into the handle 50, where it is in communication with the first air tube.

FIG. 8 also illustrates that, in embodiments, the tube set 35 may also include a video display output cable 57. The video display output cable 57 may be configured to convey image data from the image capture device 44 at the distal end 40 of the endoscope shaft 20 to, e.g., a separate video display (not shown) in response to signals generated by a user activating the buttons on the electronics control module 54. In the embodiment shown in FIG. 8, the video display output 57 is bundled into the AWS tubing set 35 along with the suction tube 362, the water tube 372, the first air tube 382 and the second air tube 383. In addition, the video display output cable 57 may also exit the handle 50 at a common location as the suction tube 362, the water tube 372, the first air tube 382 and the second air tube 383, as better shown in FIG. 7. However, it should be recognized that the video display output cable 57 may exit the handle 50 at a different location from, and may be separately disposed relative to any tube of the AWS tubing set 35, such as the suction tube 362, the water tube 372, the first air tube 382 and/or the second air tube 383, depending on, e.g., the configuration of the handle 50, the location of the various external devices (e.g., the air, suction and water sources and/or the external video display device, etc.), among other factors.

As set forth above, the water source 37 may be a water bottle 373 having a water bottle cap 390 configured to be selectively sealed onto the water bottle 373. FIG. 9 shows various features of a bottle cap 390, according to various embodiments. In the embodiment shown, the water bottle cap 390 has the water tube connection fitting 371 thereon such that the water tube 372 may be selectively connected thereto. In addition, the water bottle cap 390 may include a water suction tube 3901 that extends into the water bottle, preferably to a point near the bottom of the water bottle 373 so that the water suction tube 3901 can draw water when the water level in the water bottle 373 is relatively low. Advantageously, the water suction tube 3901 may be adjustable, e.g., extendable via a telescoping or stretching feature, such that the bottle cap 390 fits into water bottles of varying heights.

In the embodiment shown, the water bottle cap 390 also includes a second air connection fitting 3831. The second air tube connection fitting 3831 connects one end of the second air tube 383 to the water bottle 373, thereby pressurizing the interior of the water bottle 373. By pressurizing the interior of the water bottle 373, water is more forcefully and predictably pushed out of the water bottle 373 and into and through the water tube 372 to the water inlet 5222 of the water supply control valve 5221, thereby ensuring that water is available in the water supply control valve 5221 when a user presses the water supply control button 522, as shown and described hereinabove in connection with FIG. 6A.

Water bottle cap 390 also includes a pressure relief valve 3902. The pressure relief valve 3902 may function to relieve the pressure within the water bottle 373, so as to avoid damage that may be caused by over-pressurization to any components of the system, e.g., to the water bottle 373 itself, to the water bottle cap 390, to the water tube 372, and/or to any of the interior components of the handle 50 such as the water supply control valve 5221, etc. Still further, the pressure relief valve 3902 may function to help avoid injury to a patient by ensuring that the water pressure at which water is expelled from the working channel 31 of the shaft 20 and into the patient is at a level that is safe for the tissues within the patient. Of course, over-pressurization to any components of the system may be caused by a variety of different factors, e.g., by the pressure provided by the air source 38 being set too high, by there being a blockage anywhere in the system, etc.

Having the pressure relief valve 3902 be located on the water bottle cap 390 simplifies the design of the system, e.g., as compared to other systems in which a pressure relief valve is located along one of the various air or water tubes themselves. In addition, having the pressure relief valve 3902 be located on the water bottle cap 390 may help ensure that, should the pressure relief valve 3902 be activated due to an over-pressurization situation, any expelled air or water is expelled at or near the water bottle 373 which is likely to be farther away from the patient, to the surgeon, and to the other equipment, where it is less likely to cause injury or damage. Of course, the pressure relief valve 3902 may be set to relieve pressure at any one of a variety of different pressures, depending on various factors, e.g., the type of surgery being performed, the equipment being used, the age or condition of the patient, etc.

Applicant's co-pending U.S. Provisional patent application Ser. No. 18/108,558 describes various embodiments by which the second end of the second air tube 383 may be connected to a second air outlet of an air supply control valve within the handle 50. Similar such arrangements may be employed herein so as to enable the second air tube 383 to receive air from the air source 38 via the first air tube 382 and to thereby convey that air through the second air tube 383 and to the water bottle 373. For example, in one such embodiment, a second air outlet (not shown) may be located below the sealing ridge 5215 of the air valve sealing element 5214 when the air supply control valve 5211 is in the resting position, e.g., when a user has not applied to the air supply button 521 a force sufficient to overcome the air valve spring 5216. In this way, regardless of the position of the air valve sealing element 5214 (e.g., regardless of whether the user has pushed the air supply button 521), air is free to flow from the air supply source 38, through the first air supply tube 382, through the air inlet 5212 of the air supply control valve 5211, through this second air outlet of the air supply control valve 5211, through the second air tube 383, through the second air connection fitting 3831 and into the water bottle 373. In this manner, the interior of the water bottle 373 is continuously pressurized.

In still another embodiment, a configuration may be employed by which the user may selectively pressurize the interior of the water bottle 373. In such an embodiment, the second end of the second air tube 383 may be connected to a second air outlet (not shown) that is located above the sealing ridge 5215 of the air valve sealing element 5214 when the air supply control valve 5211 is in the resting position, e.g., when a user has not applied to the air supply button 521 a force sufficient to overcome the air valve spring 5216. In this way, only after a user has applied sufficient pressure on the air supply valve button 521 to overcome the biasing force of the air valve spring 5216, and thereby move the sealing ridge 5215 of the air valve sealing element 5214 to a position that is below the sealing face 5219, is air is free to flow from the air supply source 38, through the first air supply tube 382, through the first air inlet 5212 of the air supply control valve 5211, through the second air outlet of the air supply control valve 5211, through the second air tube 383, through the second air connection fitting 3831 and into the water bottle 373. In this manner, the interior of the water bottle 373 may be selectively pressurized by the user. Such selective pressurization of the water bottle 373 may be needed or desired when the level of water in the water bottle 373 is relatively low and the pressure in the water bottle 373 has decreased. Additional or alternatively, such selective pressurization of the water bottle 373 may be desirable so as to keep the pressure in the water bottle 373 low except immediately before a user desires to introduce water into the working channel 31.

FIG. 10 is a front perspective, cross-sectional view that illustrates the tubing set 35, according to various embodiments. In the embodiment shown, the tubing set 35 includes the suction tube 362, the water tube 372, the first air tube 382, the second air tube 383 and the video display output cable 57 (of course, and as mentioned previously, it should be recognized that, in alternative embodiments, the tubing set 35 may have fewer flexible tubes than those described hereinabove, may have additional flexible tubes than those flexible tubes described hereinabove, and/or may have tubes for conveying different surgical gases or fluids, e.g., saline, carbon dioxide or gas or liquid therapeutics, etc.) connected to each other by lengths of connection material 35a. Advantageously, the length (or lengths) of connection material 35a may be selectively disconnectable, e.g., able to be pulled or peeled apart or otherwise separated, by a user such that portions of the tube lengths (see length “L” in FIG. 8) may remain connected to each other by the connection material 35a while other portions of the tube lengths may be disconnected from each other. By the various tubing lengths being selectively disconnectable from each other, the various tubes in the tubing set 35 may initially be neatly bundled together, and a user may separate the various tubes from each other only as needed in order to reach the various connection points (e.g., connections to the various air, suction and/or water sources) leaving the portions that are not disconnected neatly bundled together. This minimizes the disruption or interference of unnecessarily loose tubes in the surgical space, thereby improving safety and efficiency. Of course, the location in the surgical space of the various sources, e.g., the water, air or suction sources, to which the various tubes may need to be connected may vary from surgical space to surgical space. Enabling a user to selectively disconnect only those lengths of the tubes that are needed to reach those fluid sources in their respective locations provides the user with flexibility to adjust the lengths of the various tubes that are disconnected, thereby optimizing the degree to which the various tubes are selectively disconnected from each other.

In embodiments, the lengths of connection material 35a may be part of the tubes themselves, e.g., the tubes may be collectively and simultaneously formed as a multi-lumen extrusion, with the connection lengths 35a being formed by relatively weak or separatable (e.g., weak or separatable enough to be pulled apart by a user) portions of the extrusion. Such relatively weak or separatable portions may be made so by the connection lengths 35a having, e.g., a relatively small thickness, by having perforations extending there along, or via any other configuration. Additionally or alternatively, the lengths of connection material 35a may be formed from a material that is different or separate from the materials from which the tubes themselves are formed, e.g., the connection lengths 35a may be formed from a glue, or some other tacky material, that performs the same function to keep the various tubes bundled together until a user desires to pull them apart.

It should also be noted that, in embodiments, each of the various tubes of the tubing set 35 may be connected to, and selectively disconnectable from, any one or more of the other tubes in the tubing set 35. By way of example, the water tube 372 may initially be connected to one of the other tubes (which may be any other tube), or it may be connected to more than one of such tubes. Likewise, the suction tube 362 may initially be connected to one of the other tubes (which may be any other tube), or it may be connected to more than one of such tubes, etc. The same may be true for each of the other tubes in the tubing set 35 as well. The number or type of other tubes of the tubing set 35 to which any of the given tubes of the tubing set 35 may be connected to, and selectively disconnectable from, may vary according to different factors, e.g., the total number of tubes in the tubing set 35, the size of the different tubes relative to each other, the desired difficulty in separating the tubes from each other, etc.

It should also be noted that, in certain embodiments, it may be desirable that specific ones of the tubes that are initially connected to each other by connecting lengths 35a may not be selectively disconnectable from each other. For example, in an embodiment, the water tube 372 and the second air tube 383 may not be selectively disconnectable from each other, e.g., because their distal ends are both connected to respective connection points on a bottle cap 390 that are immediately adjacent to each other. Thus, in such an embodiment, it may be desirable that the water tube 372 and the second air tube 383 are kept together along their lengths, avoiding a user unnecessarily or inadvertently disconnecting them from each other.

There are no limitations in terms of the particular embodiments described in this application, which are intended as illustrations of various aspects only. Many modifications and variations can be made without departing from its spirit and scope, as will be apparent to those skilled in the art. Functionally equivalent methods and apparatuses, in addition to those enumerated herein, will be apparent to those skilled in the art from the foregoing descriptions. Such modifications and variations are intended to fall within the scope of the appended claims. Only the terms of the appended claims are intended to be limiting, along with the full scope of equivalents to which such claims are entitled. It is also to be understood that the terminology used herein, e.g., “and”, “or”, “including”, “at least” as well as the use of plural or singular forms, etc., is for the purpose of describing examples of embodiments and is not intended to be limiting.

1. An endoscope for use in a surgical procedure, comprising:
a handle for gripping by a user;
a shaft extending from the handle, the shaft having a working channel extending longitudinally therethrough, the shaft having a distal region configured to be inserted into a patient; and
a fluid control mechanism on the handle, wherein the fluid control mechanism includes a manifold having two or more inlet openings, each one of the inlet openings configured for receiving a flow of a respective one of air, suction or water,
the manifold having a common outlet opening connected to the working channel of the shaft.

2. The endoscope of claim 1, wherein the manifold has three inlet openings, a first inlet opening configured to receive a flow of air, a second inlet opening configured to receive a flow of suction, and a third inlet opening configured to receive a flow of water.

3. The endoscope of claim 2, wherein each of the first, second and third inlet openings have a respective one of a first, second and third valve actuatable by a user to selectively control the flow of its respective one of air, suction or water.

4. The endoscope of claim 3, each of the first, second and third valves having a respective sealing element that is closed in the resting position such that, in the resting position, each valve prevents flow from the respective inlet opening to the outlet opening.

5. The endoscope of claim 4, wherein each of the first, second and third valves also include a spring that biases the valves toward their respective resting positions, each spring having a button mounted thereto that, upon being pressed by a user, overcomes the bias of the spring to move the sealing element from the closed position to an open position at which the respective inlet opening is connected to the outlet opening to thereby permit flow of the respective one of air, suction or water into and through the working channel.

6. The endoscope of claim 2, further comprising:
a tubing set connected to the handle, the tubing set including:
a first air tube connected to the first inlet opening and configured to convey air from an air source towards the working channel;
a suction tube connected to the second inlet opening and configured to convey suction in the working channel; and
a water tube connected to the third inlet opening and configured to convey water from a water source towards the working channel.

7. The endoscope of claim 6, wherein the tubing set also includes a second air tube configured to convey air from the air source to the water source so as to pressurize the water source and thereby push water from the water source towards the working channel.

8. The endoscope of claim 7, wherein the water source is a water bottle.

9. The endoscope of claim 8, wherein the water bottle has a cap that defines a first connection to the water tube and a second connection to the second air tube.

10. The endoscope of claim 1, wherein the shaft is configured for use in pediatric trans-nasal endoscopy procedures.

11. An endoscope for use in a surgical procedure, comprising:
a handle for gripping by a user;
a shaft extending from the handle, the shaft having a working channel extending longitudinally therethrough, the shaft having a distal region configured to be inserted into a patient; and
a tubing set connected to the handle, the tubing set including:
a first tube configured to convey a first one of air, suction or water to the working channel, the first tube having a first length,
a second tube configured to convey a second one of air, suction or water to the working channel, the second tube having a second length,
the first and second tube connected to each other along their respective lengths by a length of connection material, the length of connection material being selectively disconnectable by a user such that respective portions of the first and second lengths remain connected to each other by the connection material and respective other portions of the first and second lengths are disconnected from each other.

12. The endoscope of claim 11, wherein the tubing set further comprises:
a third tube having a third length,
the third tube connected to one or more of the first and second tubes along their respective lengths by a length of connection material, the length of connection material being selectively disconnectable by a user such that respective portions of the first, second and third lengths remain connected by the connection material and respective other portions of the first, second and third lengths are disconnected from each other.

13. The endoscope of claim 12, wherein the first tube is configured to convey air towards the working channel, the second tube is configured to convey suction in the working channel, and the third tube is configured to convey water towards the working channel.

14. The endoscope of claim 13, further comprising:
an air source connected to the first tube and configured to provide the air that is conveyed by the first tube, a suction source connected to the second tube and configured to provide the suction that is conveyed by the second tube,
a water source connected to the third tube and configured to provide the water that is conveyed by the third tube.

15. The endoscope of claim 14, wherein the water source is a water bottle.

16. The endoscope of claim 15, wherein the water bottle has a cap that defines a first connection to the water tube.

17. The endoscope of claim 16, wherein the tubing set further comprises:
a fourth tube having a fourth length, the fourth tube functioning as a second air tube that is configured to convey air from the air source to a second connection on the cap of the water bottle.
the fourth tube connected to at least one or more of the first, second and third tubes along their respective lengths by a length of connection material, the length of connection material being selectively disconnectable by a user such that respective portions of the first, second, third and fourth lengths remain connected by the connection material and respective other portions of the first, second, third and fourth lengths tubes are disconnected from each other.

18. The endoscope of claim 11, wherein the tubing set further comprises a video connector cable configured to conveys signals or data to an external video display device, the video connector cable having a cable length.

19. The endoscope of claim 18, wherein the video connector cable is connected to one or more of the first and second tubes along their respective lengths by a length of connection material, the length of connection material being selectively disconnectable by a user such that respective portions of the first, second and cable lengths remain connected by the connection material and respective other portions of the first, second and cable lengths are disconnected from each other.

20. (canceled)

21. An endoscope for use in a surgical procedure, comprising:
a handle for gripping by a user;
a shaft extending from the handle, the shaft having a working channel extending longitudinally therethrough, the shaft having a distal region configured to be inserted into a patient;
a water bottle having a bottle cap, the bottle cap having a first connection;
a water tube connected to the first connection of the bottle cap and to the handle so as to provide water from the water bottle to the working channel;
the bottle cap having a pressure relief valve so as to prevent over-pressurization within the water bottle, the water tube or the working channel.

22-32. (canceled)
